(19) United States
(12) Patent Application Publication
(10) Pub. No.: US 2013/0085370 A1
(43) Pub. Date: Apr. 4, 2013

(54) SYSTEMS AND METHODS FOR APPLYING AND MONITORING EYE THERAPY

(71) Applicant: Avedro, Inc., Waltham, MA (US)

(72) Inventors: Marc D. Friedman, Needham, MA (US); Evan Sherr, Ashland, MA (US); David Muller, Boston, MA (US)

(73) Assignee: Avedro, Inc., Waltham, MA (US)

(21) Appl. No.: 13/633,778

(22) Filed: Oct. 2, 2012

Related U.S. Application Data
(60) Provisional application No. 61/542,269, filed on Oct. 2, 2011.
(60) Provisional application No. 61/550,576, filed on Oct. 24, 2011.
(60) Provisional application No. 61/597,137, filed on Feb. 9, 2012.

(51) Int. Cl. A61B 3/16; A61B 3/107; A61B 6/00; A61B 3/10; A61M 37/00

(52) U.S. Cl. 600/400; 600/558; 604/20; 600/398; 600/425; 351/212; 351/205

(57) ABSTRACT

In systems and methods for generating cross-linking activity in an eye, a feedback system monitors a biomechanical strength of the eye in response to the photoactivation of a cross-linking agent applied to an eye. The feedback system includes a perturbation system that applies a force to the eye and a characterization system that determines an effect of the force on the eye. The effect of the force provides an indicator of the biomechanical strength of the eye. The characterization system determines the effect of the force on the eye by measuring an amount of deformation caused by the force or a rate of recovery from the deformation.

### CROSS REFERENCE TO RELATED APPLICATIONS

This application claims priority to U.S. Provisional Patent Application No. 61/542,269, filed Oct. 2, 2011, U.S. Provisional Patent Application No. 61/550,576, filed Oct. 24, 2011, and U.S. Provisional Patent Application No. 61/597,137, filed Feb. 9, 2012, the contents of these applications being incorporated entirely herein by reference.

### BACKGROUND OF THE INVENTION

1. Field of the Invention

The invention pertains to systems and methods for strengthening and stabilizing eye tissue, and more particularly, systems and methods for monitoring cross-linking activity during the application and activation of a cross-linking agent in corneal tissue.

2. Description of Related Art

A variety of eye disorders, such as myopia, keratoconus, and hyperopia, involve abnormal shaping of the cornea. Laser-assisted in-situ keratomileusis (LASIK) is one of a number of corrective procedures that reshape the cornea so that light traveling through the cornea is properly focused onto the retina located in the back of the eye. During LASIK eye surgery, an instrument called a microkeratome is used to cut a thin flap in the cornea. The cornea is then peeled back and the underlying cornea tissue ablated to the desired shape with an excimer laser. After the desired reshaping of the cornea is achieved, the cornea flap is put back in place and the surgery is complete.

In another corrective procedure that reshapes the cornea, thermokeratoplasty provides a noninvasive procedure that applies electrical energy in the microwave or radio frequency (RF) band to the cornea. In particular, the electrical energy raises the corneal temperature until the collagen fibers in the cornea shrink at about 60° C. The onset of shrinkage is rapid, and stresses resulting from this shrinkage reshape the corneal surface. Thus, application of energy according to particular patterns, including, but not limited to, circular or annular patterns, may cause aspects of the cornea to flatten and improve vision in the eye.

The success of procedures, such as LASIK or thermokeratoplasty, in addressing eye disorders, such as myopia, keratoconus, and hyperopia, depends on the stability of the changes in the corneal structure after the procedures have been applied.

### BRIEF SUMMARY

Embodiments according to aspects of the present disclosure provide systems and methods for strengthening and stabilizing eye tissue. In particular, systems and methods monitor cross-linking activity during the application and activation of a cross-linking agent in corneal tissue.

In some embodiments, systems and methods for generating cross-linking activity in an eye include a light source for directing light to the eye to photoactivate a cross-linking agent applied to the eye. A feedback system monitors a biomechanical strength of the eye in response to the photoactivation of the cross-linking agent. The feedback system includes a perturbation system that applies a force to the eye and a characterization system that determines an effect of the force on the eye, the effect of the force providing an indicator of the biomechanical strength of the eye. The embodiments may include a controller configured to analyze the indicator of the biomechanical strength of the eye from the feedback system; determine, based on the indicator of the biomechanical strength of the eye, the photoactivation of the the cross-linking agent; and direct the light to the eye, via the light source, to photoactivate the cross-linking agent according to a predetermined pattern. The characterization system may determine the effect of the force on the eye by measuring an amount of deformation caused by the force or a rate of recovery from the deformation.

The perturbation system may apply intraocular pressure to the eye. The perturbation system may apply acoustic or ultrasound pressure waves to the eye. The perturbation system may apply shear supersonic ultrasound to the eye. The perturbation system may include transducers configured for application to the eye. The perturbation system may include a laser system for applying laser light to the eye.

The characterization system may include a phase shift interferometer. The characterization system may include a corneal topography measurement system. The characterization system may include a Scheimpflug system. The characterization system may include an ocular coherence tomography system.

These and other aspects of the present disclosure will become more apparent from the following detailed description of embodiments of the present disclosure when viewed in conjunction with the accompanying drawings.

### BRIEF DESCRIPTION OF THE DRAWINGS

FIG. 1 provides a block diagram of an example delivery system for delivering a cross-linking agent and an activator to a cornea of an eye in order to initiate molecular cross-linking of corneal collagen within the cornea.

FIG. 2A provides a flowchart showing an example embodiment according to aspects of the present disclosure for activating cross-linking within cornea tissue using a cross-linking agent and an initiating element.

FIG. 2B provides a flowchart similar to FIG. 2A where Riboflavin may be applied topically as the cross-linking agent and UV light may be applied as the initiating element.

FIG. 3 provides an example delivery system for delivering light to the cornea 2 employing laser scanning technology.

FIG. 4 illustrates a delivery system incorporating a feedback system.

FIG. 5 illustrates alternately combinable features of an active feedback system that is operated to measure indications of biomechanical strength of eye tissue.

FIG. 6A illustrates one example of an active feedback system for the system shown generally in FIG. 5, which utilizes intraocular pressure to perturb the corneal tissue and a phase shift interferometer device to detect the resulting effect of the perturbations.

FIG. 6B illustrates one example of an active feedback system for the system shown generally in FIG. 5, which utilizes supersonic shear ultrasound waves to perturb the corneal tissue and an optical coherence tomography system to detect the resulting effect of the perturbations.

FIG. 6C illustrates one example of an active feedback system for the system shown generally in FIG. 5, which utilizes a configuration of transducers to perturb the corneal tissue and an optical coherence tomography system to detect the resulting effect of the perturbations.

FIG. 6D illustrates one example of an active feedback system for the system shown generally in FIG. 5, which utilizes a laser system to perturb the corneal tissue and an optical coherence tomography system to detect the resulting effect of the perturbations.

FIG. 7 illustrates an example configuration of transducers employed to perturb corneal tissue for measurement by an optical coherence tomography.

### DETAILED DESCRIPTION

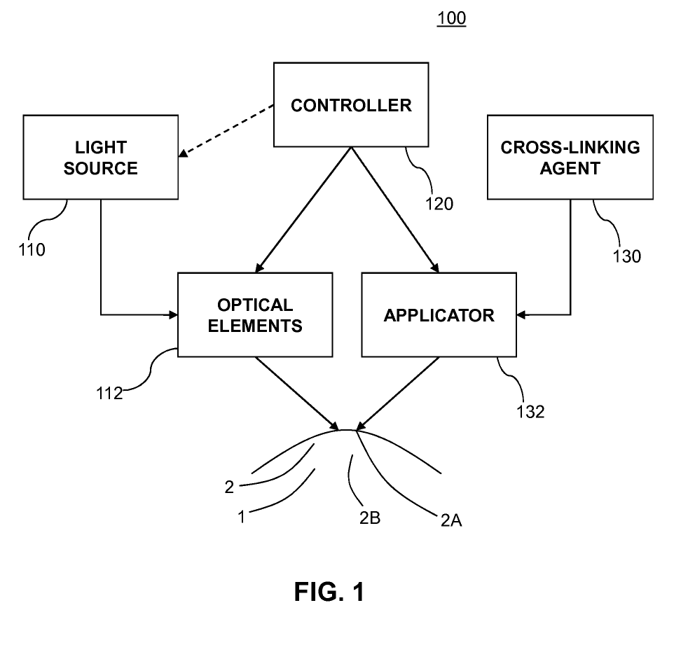

FIG. 1 provides a block diagram of an example delivery system 100 for delivering a cross-linking agent 130 and an activator to a cornea 2 of an eye 1 in order to initiate molecular cross-linking of corneal collagen within the cornea 2. Cross-linking can stabilize corneal tissue and improve its biomechanical strength. The delivery system 100 includes an applicator 132 for applying the cross-linking agent 130 to the cornea 2. The delivery system 100 includes a light source 110 and optical elements 112 for directing light to the cornea 2. The delivery system 100 also includes a controller 120 that is coupled to the applicator 132 and the optical elements 112. The applicator 132 may be an apparatus adapted to apply the cross-linking agent 130 according to particular patterns on the cornea 2 advantageous for causing cross-linking to take place within the corneal tissues. The applicator 132 may apply the cross-linking agent 130 to a corneal surface 2A (e.g., an epithelium), or to other locations on the eye 1. Particularly, the applicator 132 may apply the cross-linking agent 130 to an abrasion or cut of the corneal surface 2A to facilitate the transport or penetration of the cross-linking agent through the cornea 2 to a mid-depth region 2B.

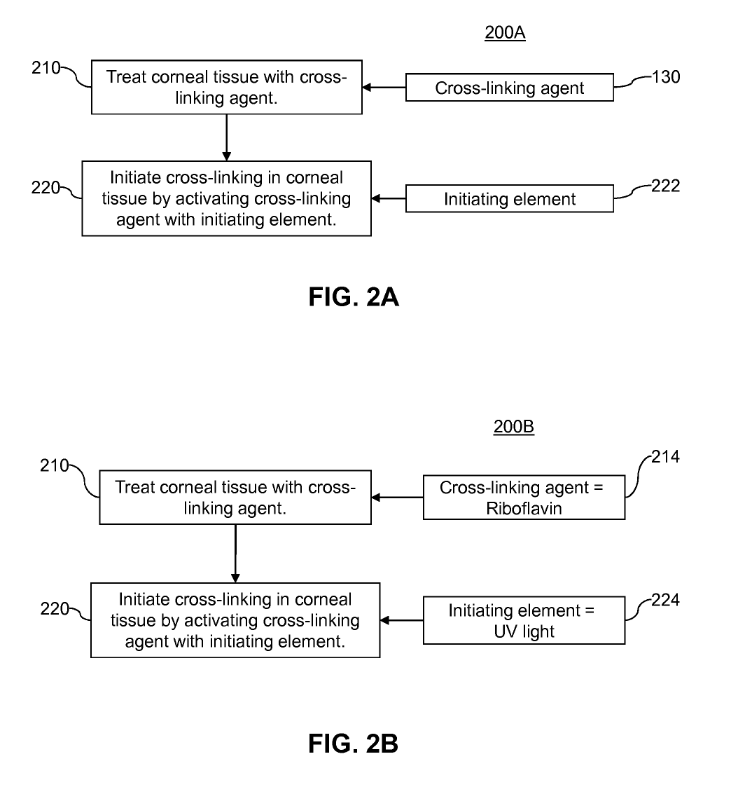

As described below in connection with FIGS. 2A-2B, which describe an exemplary operation of the delivery system 100, the cross-linking agent 130 is applied to the cornea 2 using the applicator 132. Once the cross-linking agent 130 has been applied to the cornea 2, the cross-linking agent 130 is initiated by the light source 110 (i.e., the initiating element) to cause cross-linking agent 130 to absorb enough energy to release free oxygen radicals within the cornea 2. Once released, the free oxygen radicals (i.e., singlet oxygen) form covalent bonds between corneal collagen fibrils and thereby cause the corneal collagen fibrils to cross-link and change the structure of the cornea 2. For example, activation of the cross-linking agent 130 with the light source 110 delivered to the cornea 2 through the optical elements 112 may result in cross-linking in the mid-depth region 2B of the cornea 2 and thereby strengthen and stiffen the structure of the cornea 2.

Although eye therapy treatments may initially achieve desired reshaping of the cornea 2, the desired effects of reshaping the cornea 2 may be mitigated or reversed at least partially if the collagen fibrils within the cornea 2 continue to change after the desired reshaping has been achieved. Indeed, complications may result from further changes to the cornea 2 after treatment. For example, a complication known as post-LASIK ectasia may occur due to the permanent thinning and weakening of the cornea 2 caused by LASIK surgery. In post-LASIK ectasia, the cornea 2 experiences progressive steepening (bulging).

Aspects of the present disclosure provide approaches for initiating molecular cross-linking of corneal collagen to stabilize corneal tissue and improve its biomechanical strength. For example, embodiments may provide devices and approaches for preserving the desired corneal structure and shape that result from an eye therapy treatment, such as LASIK surgery or thermokeratoplasty. In addition, aspects of the present disclosure may provide devices and approaches for monitoring the shape, molecular cross-linking, and biomechanical strength of the corneal tissue and providing feedback to a system for providing iterative initiations of cross-linking of the corneal collagen. As described herein, the devices and approaches disclosed herein may be used to preserve desired shape or structural changes following an eye therapy treatment by stabilizing the corneal tissue of the cornea 2. The devices and approaches disclosed herein may also be used to enhance the strength or biomechanical structural integrity of the corneal tissue apart from any eye therapy treatment.

Therefore, aspects of the present disclosure provide devices and approaches for preserving the desired corneal structure and shape that result from an eye treatment, such as LASIK surgery or thermokeratoplasty. In particular, embodiments may provide approaches for initiating molecular cross-linking of the corneal collagen to stabilize the corneal tissue and improve its biomechanical strength and stiffness after the desired shape change has been achieved. In addition, embodiments may provide devices and approaches for monitoring cross-linking in the corneal collagen and the resulting changes in biomechanical strength to provide a feedback to a system for inducing cross-linking in corneal tissue.

Some approaches initiate molecular cross-linking in a treatment zone of the cornea 2 where structural changes have been induced by, for example, LASIK surgery or thermokeratoplasty. However, it has been discovered that initiating cross-linking directly in this treatment zone may result in undesired haze formation. Accordingly, aspects of the present disclosure also provide alternative techniques for initiating cross-linking to minimize haze formation. In particular, the structural changes in the cornea 2 are stabilized by initiating cross-linking in selected areas of corneal collagen outside of the treatment zone. This cross-linking strengthens corneal tissue neighboring the treatment zone to support and stabilize the actual structural changes within the treatment zone.

With reference to FIG. 1, the optical elements 112 may include one or more mirrors or lenses for directing and focusing the light emitted by the light source 110 to a particular pattern on the cornea 2 suitable for activating the cross-linking agent 130. The light source 110 may be an ultraviolet light source, and the light directed to the cornea 2 through the optical elements 112 may be an activator of the cross-linking agent 130. The light source 110 may also alternatively or additionally emit photons with greater or lesser energy levels than ultraviolet light photons. The delivery system 100 also includes a controller 120 for controlling the operation of the optical elements 112 or the applicator 132, or both. By controlling aspects of the operation of the optical elements 112 and the applicator 132, the controller 120 can control the regions of the cornea 2 that receive the cross-linking agent 130 and that are exposed to the light source 110. By controlling the regions of the cornea 2 that receive the cross-linking agent 130 and the light source 110, the controller 120 can control the particular regions of the cornea 2 that are strengthened and stabilized through cross-linking of the corneal collagen fibrils. In an implementation, the cross-linking agent 130 can be applied generally to the eye 1, without regard to a particular region of the cornea 2 requiring strengthening, but the light source 110 can be directed to a particular region of the cornea 2 requiring strengthening, and thereby control the region of the cornea 2 wherein cross-linking is initiated by controlling the regions of the cornea 2 that are exposed to the light source 110.

The optical elements 112 can be used to focus the light emitted by the light source 110 to a particular focal plane within the cornea 2, such as a focal plane that includes the mid-depth region 2B. In addition, according to particular embodiments, the optical elements 112 may include one or more beam splitters for dividing a beam of light emitted by the light source 110, and may include one or more heat sinks for absorbing light emitted by the light source 110. The optical elements 112 may further include filters for partially blocking wavelengths of light emitted by the light source 110 and for advantageously selecting particular wavelengths of light to be directed to the cornea 2 for activating the cross-linking agent 130. The controller 120 can also be adapted to control the light source 110 by, for example, toggling a power switch of the light source 110.

In an implementation, the controller 120 may include hardware and/or software elements, and may be a computer. The controller 120 may include a processor, a memory storage, a microcontroller, digital logic elements, software running on a computer processor, or any combination thereof. In an alternative implementation of the delivery system 100 shown in FIG. 1, the controller 120 may be replaced by two or more separate controllers or processors. For example, one controller may be used to control the operation of the applicator 132, and thereby control the precise rate and location of the application of the cross-linking agent 130 to the cornea 2. Another controller may be used to control the operation of the optical elements 112, and thereby control with precision the delivery of the light source 110 (i.e. the initiating element) to the cornea 2 by controlling any combination of: wavelength, bandwidth, intensity, power, location, depth of penetration, and duration of treatment. In addition, the function of the controller 120 can be partially or wholly replaced by a manual operation. For example, the applicator 132 can be manually operated to deliver the cross-linking agent 130 to the cornea 2 without the assistance of the controller 120. In addition, the controller 120 can operate the applicator 132 and the optical elements 112 according to inputs dynamically supplied by an operator of the delivery system 100 in real time, or can operate according to a pre-programmed sequence or routine.

Referring to FIG. 2A, an example embodiment 200A according to aspects of the present disclosure is illustrated. Specifically, in step 210, the corneal tissue is treated with the cross-linking agent 130. Step 210 may occur, for example, after a treatment is applied to generate structural changes in the cornea and produce a desired shape change. Alternatively, step 210 may occur, for example, after it has been determined that the corneal tissue requires stabilization or strengthening. The cross-linking agent 130 is then activated in step 220 with an initiating element 222. In an example configuration, the initiating element 222 may be the light source 110 shown in FIG. 1. Activation of the cross-linking agent 130, for example, may be triggered thermally by the application of microwaves or light.

As the example embodiment 200B of FIG. 2B shows further, Riboflavin may be applied topically as a cross-linking agent 214 to the corneal tissue in step 210. As also shown in FIG. 2B, ultraviolet (UV) light may be applied as an initiating element 224 in step 220 to initiate cross-linking in the corneal areas treated with Riboflavin. Specifically, the UV light initiates cross-linking activity by causing the applied Riboflavin to release reactive oxygen radicals in the corneal tissue. In particular, the Riboflavin acts as a sensitizer to convert O₂ into singlet oxygen which causes cross-linking within the corneal tissue.

According to one approach, the Riboflavin may be applied topically to the corneal surface, and transepithelial delivery allows the Riboflavin to be applied to the corneal stroma. In general, the application of the cross-linking agent sufficiently introduces Riboflavin to mid-depth regions of the corneal tissue where stronger and more stable structure is desired.

Where the initiating element is UV light, the UV light may be generally applied to the corneal surface 2A (e.g. the epithelium) of the cornea 2 to activate cross-linking. However, regions of the cornea 2 requiring stabilization may extend from the corneal surface 2A to a mid-depth region 2B in the corneal stroma. Generally applying UV light to the corneal surface 2A may not allow sufficient penetration of the UV light to activate necessary cross-linking at a mid-depth region of the cornea. Accordingly, embodiments according to aspects of the present disclosure provide a delivery system that accurately and precisely delivers UV light to the mid-depth region 2B where stronger and more stable corneal structure is required. In particular, treatment may generate desired changes in corneal structure at the mid-depth region 2B.

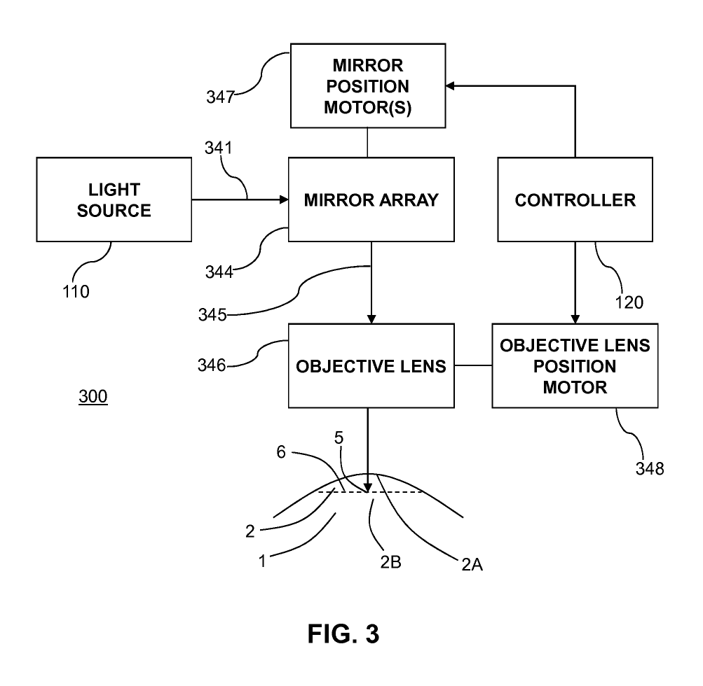

FIG. 3 provides an example delivery system adapted as a laser scanning device 300 for delivering light to the cornea 2 employing laser scanning technology. The laser scanning device 300 has the light source 110 for delivering a laser beam through an objective lens 346 into a small focal volume within the cornea 2. The laser scanning device 300 also includes the controller 120 for controlling the intensity profile of the light delivered to the cornea 2 using a mirror array 344 and for controlling the focal plane of the objective lens 346. The light source 110 can be an ultraviolet (UV) light source that emits a UV laser. A beam of light 341 is emitted from the light source 110 (e.g., UV laser) and passes to the mirror array 344. Within the mirror array 344, the beam of light 341 from the light source 110 is scanned over multiple mirrors adapted in an array. The beam of light 341 can be scanned over the mirrors in the mirror array 344 using, for example, one or more adjustable mirrors to direct the beam of light 341 to point at each mirror in turn. The beam of light 341 can be scanned over each mirror one at a time. Alternately, the beam of light 341 can be split into one or more additional beams of light using, for example, a beam splitter, and the resultant multiple beams of light can then be simultaneously scanned over multiple mirrors in the mirror array 344.

By rapidly scanning the beam of light 341 over the mirrors in the mirror array 344, the mirror array 344 outputs a light pattern 345, which has a two dimensional intensity pattern. The two dimensional intensity pattern of the light pattern 345 is generated by the mirror array 344 according to, for example, the length of time that the beam of light 341 is scanned over each mirror in the mirror array 344. In particular, the light pattern 345 can be considered a pixelated intensity pattern with each pixel represented by a mirror in the mirror array 344 and the intensity of the light in each pixel of the light pattern 345 proportionate to the length of time the beam of light 341 scans over the mirror in the mirror array 344 corresponding to each pixel. In an implementation where the beam of light 341 scans over each mirror in the mirror array 344 in turn to create the light pattern 345, the light pattern 345 is properly considered a time-averaged light pattern, as the output of the light pattern 345 at any one particular instant in time may constitute light from as few as a single pixel in the pixelated light pattern 345. In an implementation, the laser scanning technology of the delivery system 300 may be similar to the technology utilized by Digital Light Processing™ (DLP®) display technologies.

The mirror array 344 can include an array of small oscillating mirrors, controlled by mirror position motors 347. The mirror position motors 347 can be servo motors for causing the mirrors in the mirror array 344 to rotate so as to alternately reflect the beam of light 341 from the light source 340 toward the cornea 2. The controller 120 can control the light pattern 345 generated in the mirror array 344 using the mirror position motors 347. In addition, the controller 120 can control the depth within the cornea 2 that the light pattern 345 is focused to by controlling the location of the focal depth of the objective lens 346 relative to the corneal surface 2A. The controller can utilize an objective lens position motor 348 to raise and/or lower the objective lens 346 in order to adjust the focal plane 6 of the light pattern 345 emitted from the mirror array 344. By adjusting the focal plane 6 of the light pattern 345 using the objective lens motor 348, and controlling the two-dimensional intensity profile of the light pattern 345 using the mirror position motors 347, the controller 120 is adapted to control the delivery of the light source 110 to the cornea 2 in three dimensions. The three-dimensional pattern is generated by delivering the UV light to selected regions 5 on successive planes (parallel to the focal plane 6), which extend from the corneal surface 2A to the mid-depth region 2B within the corneal stroma. The cross-linking agent 130 introduced into the selected regions 5 is then activated as described above.

By scanning over selected regions 5 of a plane 6 at a particular depth within the cornea 2, the controller 120 can control the activation of the cross-linking agent 130 within the cornea 2 according to a three dimensional profile. In particular, the controller 120 can utilize the laser scanning technology of the laser scanning device 300 to strengthen and stiffen the corneal tissues by activating cross-linking in a three-dimensional pattern within the cornea 2. In an implementation, the objective lens 346 can be replaced by an optical train consisting of mirrors and/or lenses to properly focus the light pattern 345 emitted from the mirror array 344. Additionally, the objective lens motor 348 can be replaced by a motorized device for adjusting the position of the eye 1 relative to the objective lens 346, which can be fixed in space. For example, a chair or lift that makes fine motor step adjustments and adapted to hold a patient during eye treatment can be utilized to adjust the position of the eye 1 relative to the objective lens 346.

Advantageously, the use of laser scanning technologies allows cross-linking to be activated beyond the corneal surface 2A of the cornea 2, at depths where stronger and more stable corneal structure is desired, for example, where structural changes have been generated by an eye therapy treatment. In other words, the application of the initiating element (i.e., the light source 110) is applied precisely according to a selected three-dimensional pattern and is not limited to a two-dimensional area at the corneal surface 2A of the cornea 2.

Although the embodiments described herein may initiate cross-linking in the cornea according to an annular pattern defined, for example, by a thermokeratoplasty applicator, the initiation pattern in other embodiments is not limited to a particular shape. Indeed, energy may be applied to the cornea in non-annular patterns, so cross-linking may be initiated in areas of the cornea that correspond to the resulting non-annular changes in corneal structure. Examples of the non-annular shapes by which energy may be applied to the cornea are described in U.S. patent Ser. No. 12/113,672, filed on May 1, 2008, the contents of which are entirely incorporated herein by reference.

Some embodiments may employ Digital Micromirror Device (DMD) technology to modulate the application of initiating light, e.g., UV light, spatially as well as a temporally. Using DMD technology, a controlled light source projects the initiating light in a precise spatial pattern that is created by microscopically small mirrors laid out in a matrix on a semiconductor chip, known as a (DMD). Each mirror represents one or more pixels in the pattern of projected light. The power and duration at which the light is projected is determined as described elsewhere.

Embodiments may also employ aspects of multiphoton excitation microscopy. In particular, rather than delivering a single photon of a particular wavelength to the cornea 2, the delivery system (e.g., 100 in FIG. 1) delivers multiple photons of longer wavelengths, i.e., lower energy, that combine to initiate the cross-linking. Advantageously, longer wavelengths are scattered within the cornea 2 to a lesser degree than shorter wavelengths, which allows longer wavelengths of light to penetrate the cornea 2 more efficiently than shorter wavelength light. For example, in some embodiments, two photons may be employed, where each photon carries approximately half the energy necessary to excite the molecules in the cross-linking agent 130 that release oxygen radicals. When a cross-linking agent molecule simultaneously absorbs both photons, it absorbs enough energy to release reactive oxygen radicals in the corneal tissue. Embodiments may also utilize lower energy photons such that a cross-linking agent molecule must simultaneously absorb, for example, three, four, or five, photons to release a reactive oxygen radical. The probability of the near-simultaneous absorption of multiple photons is low, so a high flux of excitation photons may be required, and the high flux may be delivered through a femtosecond laser. Because multiple photons are absorbed for activation of the cross-linking agent molecule, the probability for activation increases with intensity. Therefore, more activation occurs where the delivery of light from the light source 110 is tightly focused compared to where it is more diffuse. The light source 110 may deliver a laser beam to the cornea 2. Effectively, activation of the cross-linking agent 330 is restricted to the smaller focal volume where the light source 310 is delivered to the cornea 2 with a high flux. This localization advantageously allows for more precise control over where cross-linking is activated within the cornea 2.

Referring again to FIG. 1, embodiments employing multiphoton excitation microscopy can also optionally employ multiple beams of light simultaneously applied to the cornea 2 by the light source 110. For example, a first and a second beam of light can each be directed from the optical elements 112 to an overlapping region of the cornea 2. The region of intersection of the two beams of light can be a volume in the cornea 2 where cross-linking is desired to occur. Multiple beams of light can be delivered to the cornea 2 using aspects of the optical elements 112 to split a beam of light emitted from the light source 310 and direct the resulting multiple beams of light to an overlapping region of the cornea 2. In addition, embodiments employing multiphoton excitation microscopy can employ multiple light sources, each emitting a beam of light that is directed to the cornea 2, such that the multiple resulting beams of light overlap or intersect in a volume of the cornea 2 where cross-linking is desired to occur. The region of intersection may be, for example, in the mid-depth region 2B of the cornea 2, and may be below the corneal surface 2A. Aspects of the present disclosure employing overlapping beams of light to achieve multi-photon microscopy may provide an additional approach to controlling the activation of the cross-linking agent 130 according to a three-dimensional profile within the cornea 2.

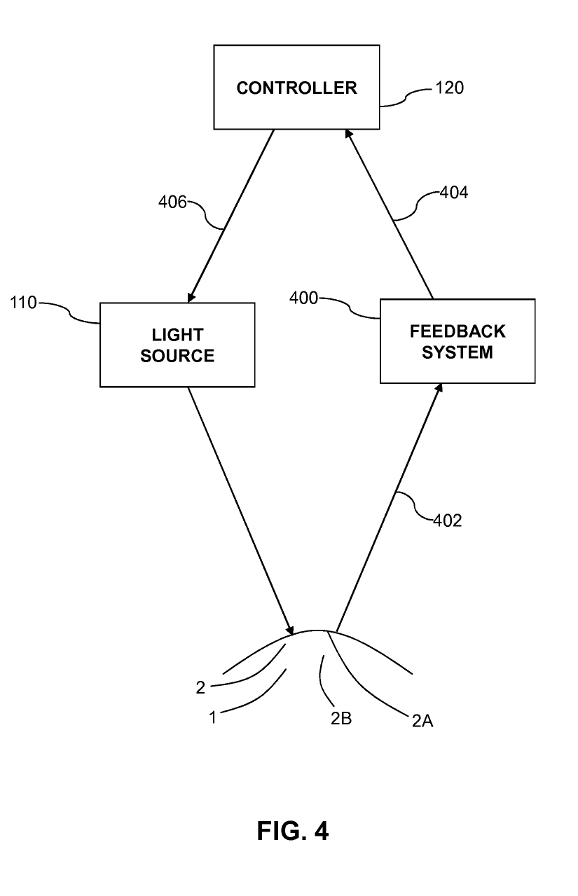

Aspects of the present disclosure, e.g., adjusting the parameters for delivery and activation of the cross-linking agent, can be employed to reduce the amount of time required to achieve the desired cross-linking. In an example implementation, the time can be reduced from minutes to seconds. While some configurations may apply the initiating element (i.e., the light source 110) at a flux dose of 5 J/cm², aspects of the present disclosure allow larger doses of the initiating element, e.g., multiples of 5 J/cm², to be applied to reduce the time required to achieve the desired cross-linking. Highly accelerated cross-linking is particularly possible when using laser scanning technologies (such as in the delivery system 300 provided in FIG. 3) in combination with a feedback system 400 as shown in FIG. 4, such as a rapid video eye-tracking system, described below.

To decrease the treatment time, and advantageously generate stronger cross-linking within the cornea 2, the initiating element (e.g., the light source 110 shown in FIG. 1) may be applied with a power between 30 mW and 1 W. The total dose of energy absorbed in the cornea 2 can be described as an effective dose, which is an amount of energy absorbed through a region of the corneal surface 2A. For example the effective dose for a region of the cornea 2 can be, for example, 5 J/cm², or as high as 20 J/cm² or 30 J/cm². The effective dose delivering the energy flux just described can be delivered from a single application of energy, or from repeated applications of energy. In an example implementation where repeated applications of energy are employed to deliver an effective dose to a region of the cornea 2, each subsequent application of energy can be identical, or can be different according to information provided by the feedback system 400.

Treatment of the cornea 2 by activating cross-linking produces structural changes to the corneal stroma. In general, the optomechanical properties of the cornea changes under stress. Such changes include: straightening out the waviness of the collagen fibrils; slippage and rotation of individual lamellae; and breakdown of aggregated molecular superstructures into smaller units. In such cases, the application of the cross-linking agent 130 introduces sufficient amounts of cross-linking agent to mid-depth regions 2B of the corneal tissue where stronger and more stable structure is desired. The cross-linking agent 130 may be applied directly to corneal tissue that have received an eye therapy treatment and/or in areas around the treated tissue.

To enhance safety and efficacy of the application and the activation of the cross-linking agent, aspects of the present disclosure provide techniques for real time monitoring of the changes to the collagen fibrils with a feedback system 400 shown in FIG. 4. These techniques may be employed to confirm whether appropriate doses of the cross-linking agent 130 have been applied during treatment and/or to determine whether the cross-linking agent 130 has been sufficiently activated by the initiating element (e.g., the light source 110). General studies relating to dosage may also apply these monitoring techniques.

Moreover, real time monitoring with the feedback system 400 may be employed to identify when further application of the initiating element (e.g., the light source 110) yields no additional cross-linking. Where the initiating element is UV light, determining an end point for the application of the initiating element protects the corneal tissue from unnecessary exposure to UV light. Accordingly, the safety of the cross-linking treatment is enhanced. The controller 120 for the cross-linking delivery system can automatically cease further application of UV light when the real time monitoring from the feedback system 400 determines that no additional cross-linking is occurring.

FIG. 4 illustrates a delivery system incorporating the feedback system 400. The feedback system 400 is adapted to gather measurements 402 from the eye 1, and pass feedback information 404 to the controller 120. The measurements 402 can be indicative of the progress of strengthening and stabilizing the corneal tissue. The measurements 402 can also provide position information (“targeting information”) regarding the location of the eye 1 and can detect movement of the cornea 2, and particularly the regions of the corneal tissue requiring stabilization. The feedback information 404 is based on the measurements 402 and provides input to the controller 120. The controller 120 then analyzes the feedback information 404 to determine how to adjust the application of the initiating element, e.g., the light source 110, and sends command signals 406 to the light source 110 accordingly. Furthermore, the delivery system 100 shown in FIG. 1 can be adapted to incorporate the feedback system 400 and can adjust any combination of the optical elements 112, the applicator 132, or the light source 110 in order to control the activation of the cross-linking agent 130 within the cornea 2 based on the feedback information 404 received from the feedback system 400.

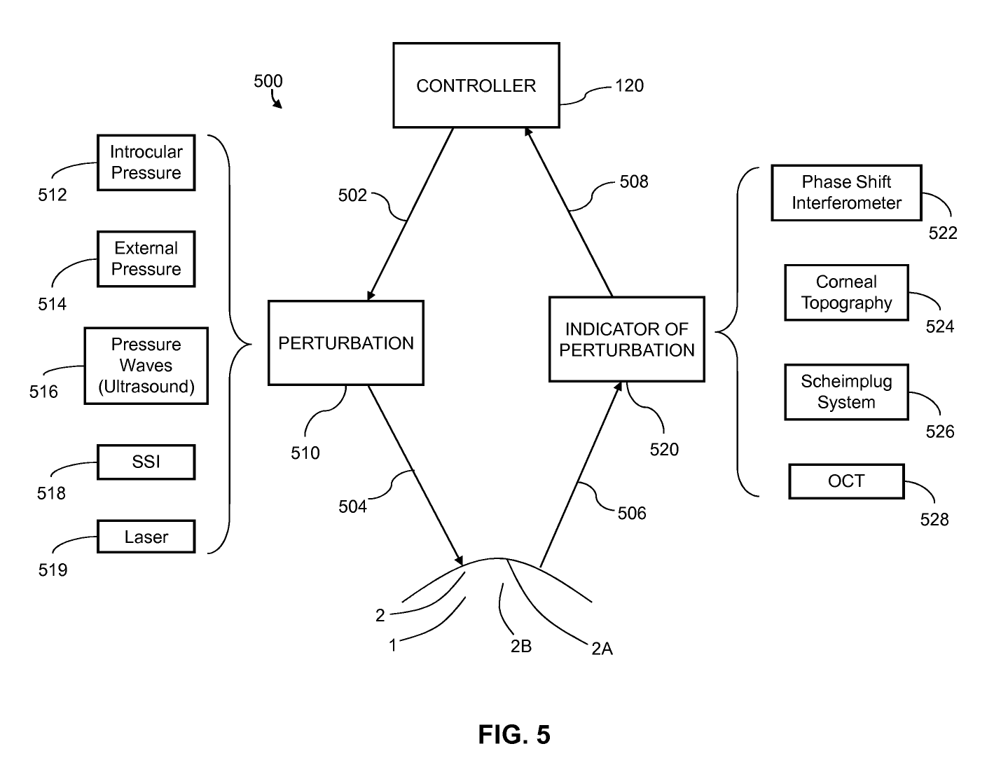

FIG. 5 illustrates alternately combinable features of an active feedback system 500 that is operated to measure indications of biomechanical strength of eye tissue. In an implementation, the controller 120 is configured to operate a perturbation source 510 to disturb the eye 1, while simultaneously observing effect of the perturbation on the eye via a perturbation indication characterization system 520. As will be described further herein, both the perturbation source 510 and the perturbation indication characterization system 520 can be implemented with a variety of technologies. In general, however, the perturbation system 510 introduces some physical force (i.e., a perturbation) on the cornea 2 that causes the cornea 2 to temporarily deform. The effect of the perturbation is then characterized by measuring, for example, the amount of deformation and/or rate of recovery of the cornea 2, using the characterization system 520. In some implementations, the characterization system 520 provides indications of the perturbation with sufficient resolution across the cornea 2, and through its depth, that a three dimensional model (“mapping”) of corneal strength can be developed.

Generally then, the controller 120 sends command signals 502 to the perturbation source 510 to instruct the perturbation source 510 to provide a standardized perturbation to the cornea 2. The physical influence 504 is then imparted on the cornea 2 by the perturbation source 510 so as to cause the cornea 2 to be deflected, deformed, or otherwise disturbed. Indicators 506 of the perturbation are then received by the characterization system 520 and data signals 508 based on the indicators 508 are then passed back to the controller 120 for additional processing.

As shown in FIG. 5, the perturbation source 510 can be implemented as time changing intraocular pressure 512. It is specifically noted that in the particular example where the perturbation source 510 is implemented as time changing intraocular pressure 512, command signals 502 are not sent to the perturbation source 510. The time-changing intraocular pressure 512 perturbs the cornea 2 by modifying the pressure experienced by the back side of the eye 1 over the course of a cardiac pulse cycle. While the changes in intraocular pressure (e.g., from minimum back pressure on the eye 1 to maximum back pressure on the eye 1) are not generally standardized amongst different individuals, the pressure changes can provide a relatively stable physical influence 504 for each individual. Thus comparisons of the amount of deflection over the course of a cardiac pulse cycle on a particular individual before and after initiation of cross linking can provide a useful indicator 506 of the corneal biomechanical strength of the individual.

The perturbation source 510 can also be a source of external pressure 514, such as from a contacting object designed to apply a standard force 504 to the corneal surface 2A or such as from a controllable stream of air which creates a standard force 504 on the cornea 2. The perturbation source 510 can also be implemented as a pressure wave directed at the corneal tissue 2. The pressure wave can be, for example, acoustic and/or ultrasound pressure waves 504 that can be generally applied to the corneal tissue 2 or can be focused at particular regions. Additionally or alternatively, the perturbation source 510 can be implemented as supersonic ultrasound waves 518 propagating in a shear direction through the cornea 2. For example, the shear supersonic ultrasound waves can be generated by a system utilized in supersonic shear imaging (“SSI”) systems. An example implementation of an SSI system useful in generating shear supersonic ultrasound waves to provide the physical force 504 to perturb the cornea 2 is described in, for example, M. Tanter et al., High-Resolution Quantitative Imaging of Cornea Elasticity Using Supersonic Shear Imaging, IEEE Transactions on Medical Imaging, vol. 28, no. 12, Dec. 2009, pp. 1881-1893, the contents of which are hereby incorporated entirely herein by reference.

The characterization system 520 can also be implemented by a variety of different technologies. For example, the characterization system 520 can be implemented as a phase shift interferometer 522. The phase shift interferometer 522 utilizes polarized coherent light beams that are interfered with one another and the resulting interference patterns are captured in an image capture system (“camera”). One beam of light is reflected from the corneal surface 2A while the other, interfering beam of light is reflected from a reference surface and the interference patterns thus provide indications of the differences in the surface between the reference surface and the corneal surface 2A. Rapid measurements 506 of the corneal surface 2A following and/or simultaneous with the application of the perturbation 504 thus provide an indication of the biomechanical response of the corneal tissue 2 to the perturbation 504. Examples of phase shift interferometric systems for use in dynamically characterizing a corneal surface are provided in commonly assigned U.S. patent application Ser. No. 13/051,699, filed Mar. 18, 2011, the contents of which are hereby incorporated entirely herein by reference.

In addition, the characterization system 520 can be implemented as a corneal topography measurement system 524, such as a wavefront detection system. Similar to the phase shift interferometer 522, a topography measurement system 524 can characterize the biomechanical strength of the cornea 2 by dynamically monitoring corneal surface 2A topography to characterize the amount of motion of the cornea 2 resulting from the perturbation 504.

The characterization system 520 can also be implemented as a Scheimpflug system 526 configured to acquire a series of cross-sectional images eye 1. The Scheimpflug system 526 utilizes a slit of light for illumination of the corneal tissue 2. Scheimpflug imaging differs from conventional techniques in that the object plane, lens plane, and image plane are not parallel to each other, but intersect in a common straight line. A major advantage of the Scheimpflug geometry is that a wide depth of focus is achieved. The Scheimpflug principle has been applied in ophthalmology to obtain optical sections of the entire anterior segment of the eye 1, from the anterior surface of the cornea 2 to the posterior surface of the lens. This type of imaging allows assessment of anterior and posterior corneal topography, anterior chamber depth, as well as anterior and posterior topography of the lens. Several commercial ophthalmic Scheimpflug systems are available today. These include the Pentacam corneal topography system made by Oculus (http://www.pentacam.com/sites/messprinzip.php) as well as the GALILEI and GALILEI G2 corneal topography systems made by Zeimer Group (http://www.ziemergroup.com/products/g2-main.html).

The characterization system 520 can also be implemented as an ocular coherence tomography system 528 (“OCT”). The OCT system 528 generally utilizes low coherence interferometry of white optical light or near-infrared light. By contrast with coherent interferometry techniques with long coherence lengths (e.g., those utilizing laser light sources), in the OCT system 528, interference is shortened to a distance of micrometers, due to the use of broadband light sources (e.g., sources that can emit light over a broad range of frequencies). Light in the OCT system is broken into two beams—a sample beam, which is directed toward the cornea 2, and a reference beam, which is directed toward a reference surface. The combination of reflected light from the cornea and the reference surface are interfered to produce an interference pattern. Constructive interference generally occurs only if light from the two beams travel an optical distance within a coherence length. By scanning the reference surface (e.g., a reference mirror) a reflectivity profile of the cornea can be obtained at different depths of the corneal tissue 2. Generally, areas of the cornea 2 that reflect back a significant amount of light will create greater interference than areas that do not. Any light that is outside the short coherence length will not interfere. Thus, adjusting the reference surface allows the OCT system 528 to be tuned to particular depths of the cornea 2. Such a reflectivity profile (“interference pattern”) is referred to as an A-scan. These axial depth scans (A-scans) can be laterally combined to create a cross-sectional tomography (B-scan). The OCT system 528 thus provides a high resolution (micrometer scale) three-dimensional (to millimeter depths) profile of the corneal tissue 2.

While the OCT system 528 is described above as a time domain OCT, which scans varying depths of the cornea 2 during distinct time intervals, this is for illustrative purposes only. It is specifically noted that the OCT system 528 can be implemented as one of a variety of available OCT systems, including frequency domain OCT, spectral domain OCT, Fourier domain OCT, time encoded frequency domain OCT, and swept source OCT. Generally, a frequency domain OCT system operates by performing Fourier transforms on the received data to identify the contributions from the returning signal corresponding to different depths in the corneal tissue 2. A frequency domain OCT generally is able to generate a full three-dimensional model of the eye 1 in less time compared to a time domain OCT, because the position of the reference arm is not adjusted. Frequency domain OCT systems can be implemented with spatially encoded detectors utilizing, for example, gratings situated in front of CCD detector arrays to distinctly detect different wavelengths of the returning signal via different regions of the CCD detector array. Time encoded frequency domain OCT are implemented with a reference light source that has a characteristic frequency which changes in time. Thus, in a time encoded frequency domain OCT, the cornea 2 is probed according to varying wavelengths of light, and the returning signals therefore correspond to varying depths of the corneal tissue 2.

The various implementations of the OCT system 528 offer different performance criteria in the form of scan depth, axial resolution, speed of measurement, and signal to noise ratio. These performance criteria may influence a designer's choice of system. For example, implementing the OCT system 528 as a frequency domain OCT system may be desirable because a frequency domain OCT system offers enhanced measurement speed and can generate a full three-dimensional model of the cornea 2 without modifying physical features of the OCT system 528 (such as the position of the reference surface). However, the various OCT systems each are operable to generate three-dimensional profiles of the cornea 2.

Dynamically gathering three-dimensional profiles of the cornea 2 using the OCT system 528 allows the effect of the perturbation 504 to be precisely characterized at a high resolution. For example, the corneal tissue can be characterized by a plurality of connected micrometer scale volumetric regions, and the displacement of each volumetric region from a nominal starting position to a maximum displacement position can be measured as a result of the perturbation 504 acting on the eye 1. The amount of displacement of each segment of the cornea 2 can thus provide an indication of the biomechanical strength of the corneal tissue 2 at a micrometer scale. An example of an OCT system is the Stratus OCT™ (Carl Zeiss Meditec, Inc.).

Furthermore, the characterization system 520 can be implemented as an Ocular Response Analyzer for measuring corneal hysteresis in response to a changing optical pressure, available from Reichert, Inc., and as described in Michael Sullivan-Mee, The Role of Ocular Biomechanics In Glaucoma Management, Review of Optometry, Oct. 15, 2008, pp. 49-54, the contents of which are incorporated herein by reference in their entirety.

It is specifically noted that any combination of the various disclosed perturbation sources 510 (e.g., the intraocular pressure 512, the external pressure source(s) 514, the pressure waves 516, and/or the shear supersonic ultrasound waves 518) can be combined with any combination of the various disclosed characterization systems 520 (e.g., the phase shift interferometer 522, the corneal topography system 524, the Scheimpflug system 526, and/or the ocular coherence tomography system 528). Furthermore, the present disclosure is not limited to the particular examples (512, 514, 516, 518, 519) of the perturbation sources 510 disclosed herein, nor is the present disclosure limited to the particular examples (522, 524, 526, 528) of the characterization systems 520 disclosed herein.

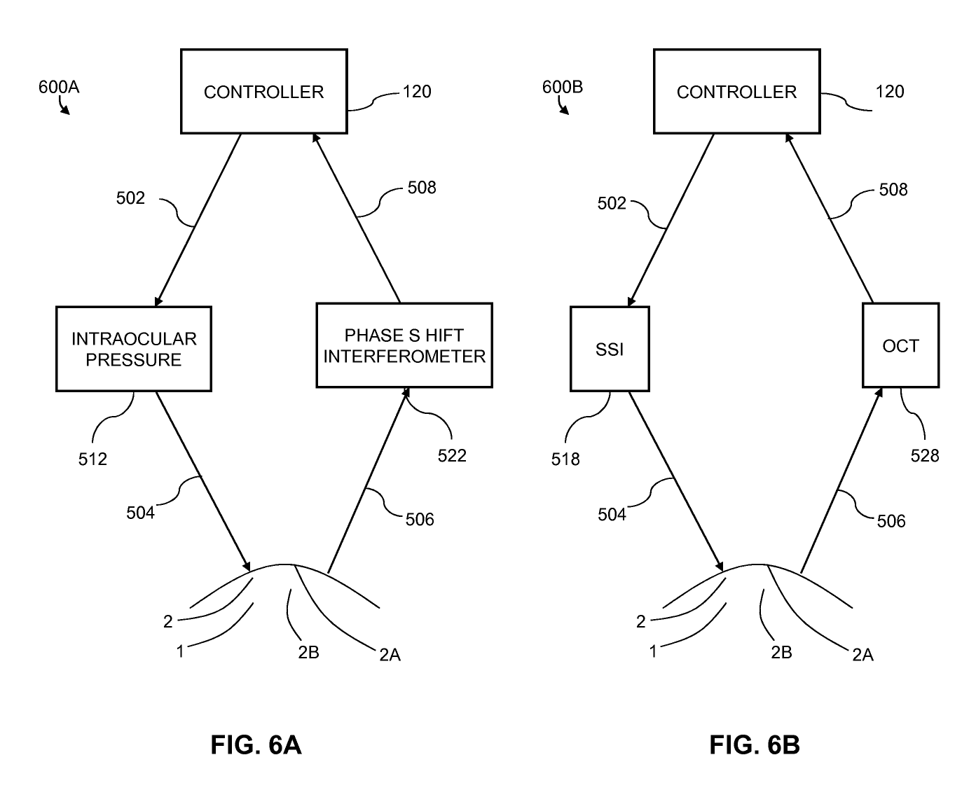

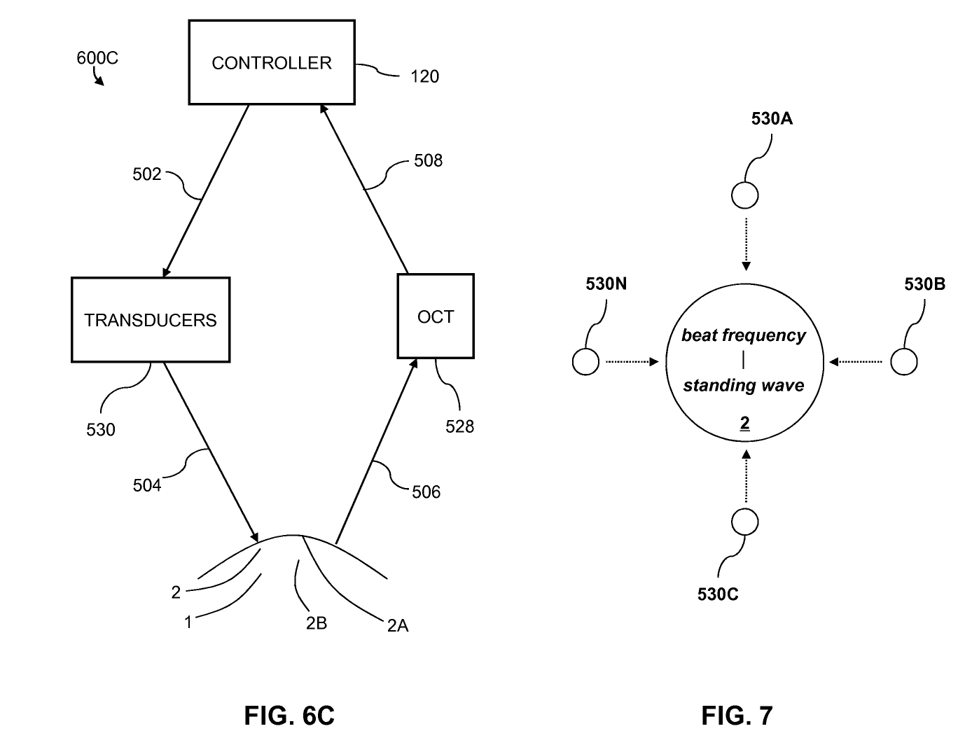

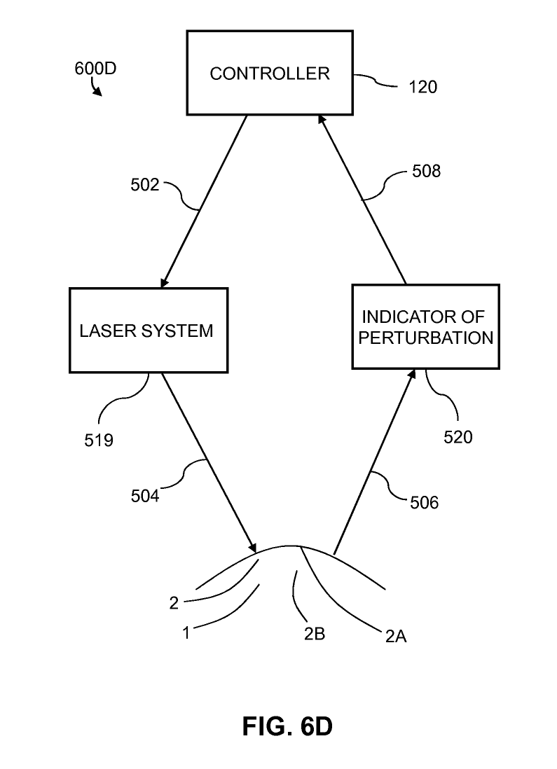

FIG. 6A illustrates one example of an active feedback system 600A for the system 500 shown generally in FIG. 5. The system 600A utilizes intraocular pressure 512 to perturb the corneal tissue 2 and a phase shift interferometer 522 device to detect the resulting effect of the perturbations. The feedback system 600A advantageously does not rely on an artificial means for the external force 504 applied to perturb the eye 1. The eye 1 is perturbed by the force of the modulations of the intraocular pressure 512 alone. In addition, the phase shift interferometer operates at a high rate to capture many topographic profiles of the corneal surface 2A over the course of a single cardiac pulse cycle.

FIG. 6B illustrates another example of an active feedback system 600B for the system 500 shown generally in FIG. 5. The system 600B utilizes supersonic shear ultrasound waves 518 (e.g., generated by an SSI system) to perturb the corneal tissue 2 and an optical coherence tomography system 528 to detect the resulting effect of the perturbations. It is particularly noted that the perturbations 504 from the SSI system 518 generate typical displacements of the corneal tissue 2 on the order of a micron, while the OCT system 528 monitoring the displacements 506 of the cornea 2 is advantageously configured to be able to resolve structural features of the corneal tissue 2 on the order of a micrometer. The high resolution (e.g., micrometer scale) of both the SSI 518 and OCT 528 systems allow for similarly high resolution characterization of the corneal biomechanical properties. In addition, the OCT system 528 offers penetration depth of as much as a few millimeters to provide indications of the biomechanical properties of the cornea 2 at depths below the corneal surface 2A.

FIG. 6C illustrates yet another example of an active feedback system 600C for the system 500 shown generally in FIG. 5. The system 600C utilizes a configuration of transducers 530 to perturb the corneal tissue 2 and an optical coherence tomography system 528 to detect the resulting effect of the perturbations. FIG. 7 illustrates an example configuration of a plurality of ultrasonic transducers 530A-N arranged along the edges of the corneal tissue 2. It is understood that any number of transducers 530 may be employed at any location(s) about the corneal tissue 2 to achieve the desired perturbation. The transducers 530, for example, may be assembled in a device that resembles a contact lens and allows the transducers to deliver ultrasonic signals to the eye 1. Such micro transducer systems may be provided, for example, by Sensimed AG (Lausanne, Switzerland). The controller 120 activates the transducers 530 to produce signals that perturb the corneal tissue 2. As shown in FIG. 7, the transducers 530A-N are activated in a sequence that produces a beat frequency through ultrasonic signals. The beat frequency in turn produces a standing wave in the corneal tissue 2, which can be measured with the OCT system 528 in a manner similar to the embodiments described above.

FIG. 6D illustrates yet another example of an active feedback system 600D for the system 500 shown generally in FIG. 5. The system 600C utilizes a laser system 519 to perturb the corneal tissue 2 and a characterization system 520, as described herein. To apply a perturbation force, the laser system 519 may include any laser which is capable of being sufficiently absorbed by the corneal tissue 2 in a manner that is fast enough to elicit a shock wave or similar tissue deformation. In example embodiments, the laser system 519 may employ an excimer laser, an erbium YAG laser, or a femtosecond laser. It is understood, however, that other lasers with similar characteristics may be employed for stress wave generation.

Excimer lasers produce very short pulses (e.g., approximately 1 to 50 nsec) with high peak power and are capable of producing the necessary shock waves. Excimer lasers capable of producing the desired excitation include those having wavelengths of approximately 193-308 nm. For example, an excimer laser with a wavelength of approximately 193 nm results in high absorption in tissue while limiting penetration to typically less than a micron. Indeed, excimer laser ablation is known to produce mechanical waves which propagate along the surface of and through tissue. The mechanics of these induced waves have been well characterized for the cornea. Broad beam excimer lasers are capable of producing pressure waves of tens of atmospheres in ocular tissue and are capable of inducing stress on ocular tissue.

Thus, in one example embodiment, a material of known viscosity, such as drops of methylcellulose, is applied to the cornea with a thickness of, e.g., approximately 46 microns. In one aspect, the layer protects the cornea during application of the excimer laser. An excimer laser, e.g., an excimer laser having a wavelength of approximately 195 nm, ablates the layer, and a deforming force is correspondingly applied to the cornea. With a known application interval, power, wavelength, etc., the force applied to the cornea can be determined from the rate of ablation. The translucence of the layer allows techniques, such as OCT, to be used to characterize the effect of the force applied by the excimer laser via the layer.

Erbium YAG lasers are solid-state lasers whose lasing medium is erbium-doped yttrium aluminium garnet. In particular, at a wavelength of approximately 2940 nm an erbium YAG laser is strongly absorbed by water. This confines the absorption to a tissue layer that is approximately 5 to 20 microns thick (fluence dependent). With pulse durations typically around 50-250 μs because of its absorption, the erbium YAG laser is capable of producing stress waves in tissue similar to those seen with an excimer laser. Like excimer lasers, erbium lasers are surface absorbed; hence, stress wave propagation originates on the surface and propagate through the tissue as well as circumferentially (similar to ripples in a pond).

Femtosecond lasers have a very short wavelength, allowing for ablation and shockwave generation by focusing energy into a very small area of high peak power. A wavelength that may be employed for corneal use is approximately 1053 nm. This wavelength is not highly absorbed but high focus and short, high peak power pulses allow for creation of plasma and associated stress waves.

The active feedback system 600D controls the positioning of laser focus. In some embodiments, a large spot is placed substantially coincident with the area to be measured. A broad beam is employed over the cornea to apply a relatively uniform pressure to the tissue, compressing and/or deflecting it. A large spot allows more energy to be applied, hence causing greater deformation.

In other embodiments, a large spot is placed adjacent to the area to be measured. A broad beam is employed adjacent to the region of interest to produce a wave that propagates transversely across the cornea. This approach may measure tissue or surface-wave time of flight across the material. Adjacent application may mean applying the beam on the same tissue out of the area of measurement (such as on the sclera for corneal measurement) or on an adjacent structure which is acoustically communicating with the target tissue (like on the orbital rim to propagate to the eye).

In further embodiments, a single small spot is placed either coincident or adjacent to the area to be measured. A focal stress is applied to propagate like ripples from a pebble in the water through surrounding tissue.

In yet further embodiments, a line or array of small spots placed either coincident or adjacent to the area to be measured locally excites tissue for local measurements which may be used to determine an overall map of tissue mechanics.

The physical application of the lasers may be sequentially patterned or randomly patterned. In addition, the lasers may be physically applied with varying energy.

The laser system 519 in the active feedback system 600D may apply the lasers according to various beam shapes. For example, the lasers may be applied as broad, disk-shapes; small, local points; annulus shapes (for evaluating inward, outward and or intra-annular wave propagation); lines; ellipses; spirals; rectangles, triangles; or any combination thereof. The beam shapes may increase or decrease in height, width, length, diameter, rotation, or any combination thereof.

The application of the lasers through the active feedback system 600D is also temporally controlled. In some embodiments, the laser system 519 applies a single pulse. Short pulsed laser ablation can be approximated as an impulse or delta function. This causes a stress impulse and accompanying tissue displacement. Propagation of displacement follows visco-elastic deformation (underdamped, critically damped or overdamped) whose mechanics can be defined by appropriate methods and analysis.

In other embodiments, the lasers are applied as sequential array of pulses to build a regional map or induce composite wave propagation. In further embodiments, the lasers are applied as pulses in a rhythmic fashion tuned to the harmonics of the tissue to build a standing or propagating acoustic wave in the tissue. In yet other embodiments, the lasers are applied according to a burst of pulses to approximate a step, ramp or other function, where the pulse burst is fast enough to damp out refraction between pulses. In additional embodiments, the lasers may be applied with varying pulse duration and/or varying duty cycle.

The propagation of deformation can be evaluated in the time domain, evaluating stress-strain relationship due to impulse or pseudo-continuous pressure. Additionally or alternatively, deformations caused by impulse, pseudo-continuous and harmonic impulse may be converted to frequency domain with the fast Fourier transform (FFT) of the temporal data. In frequency domain, determination of elastic and viscous parameters are more readily calculated.

As described above, the active feedback systems 500, 600A, 600B, 600C, and 600D can be utilized to characterize biomechanical properties (e.g., corneal strength, rigidity) of the cornea 2 prior to, after, and/or during initiating cross-linking treatment. In implementations where cross-linking therapy is dynamically adjusted according to the data signals 508, the controller 120 can be adapted to determine the amount of change (“outcome”) in biomechanical properties in the cornea 2 following an in initial cross-linking treatment, and to determine a subsequent dose of cross-linking initiation based on the first outcome. The dose can be characterized by the energy level of the initiating element, the power of the initiating element, the concentration and/or amount of the cross-linking agent, the intensity pattern and/or duration of the application of the initiating element, and/or any combination of these.

Generally, the cross-linking agent 122 may be applied to the corneal tissue in an ophthalmic solution, e.g., in the form of eye drops. In some cases, the cross-linking agent 122 is effectively applied to the corneal tissue by removing the overlying epithelium before application. However, in other cases, the cross-linking agent 122 is effectively applied in a solution that transitions across the epithelium into the underlying corneal tissue, i.e., without removal of the epithelium. For example, a transepithelial solution may combine Riboflavin with approximately 0.1% benzalkonium chloride (BAC) in distilled water. Alternatively, the transepithelial solution may include other salt mixtures, such as a solution containing approximately 0.4% sodium chloride (NaCl) and approximately 0.02% BAC. Additionally, the transepithelial solution may contain methyl cellulose, dextran, or the like to provide a desired viscosity that allows the solution to remain on the eye for a determined soak time.

Although embodiments of the present disclosure may describe stabilizing corneal structure after treatments, such as LASIK surgery and thermokeratoplasty, it is understood that aspects of the present disclosure are applicable in any context where it is advantageous to form a stable three-dimensional structure of corneal tissue through cross-linking.

As described above, OCT systems may be employed to generate three-dimensional profiles of the cornea. OCT systems can also be used to develop maps of epithelial thickness. Such OCT systems provide sufficient resolution to distinguish the epithelium from the stroma. The information regarding the epithelial thickness may be used in combination with corneal thickness and topography maps as provided by Scheimpflug, OCT, or other similar systems. This combined information on the epithelial thickness, stroma thickness, as well as topography maps of the anterior and posterior sections of the cornea. This combined information may be used by physicians to create a pre-treatment plan for an individual patient. This is especially advantageous for trans-epithelial. Since there is often epithelial thickness variation in keratoconus. By knowing these variations upfront, the pre-treatment plan is more exacting to the attempted correction. In some embodiments, fluorescence dosimetry may be employed to measure how the actual trans-epithelial formulation is diffusing through the epithelial. Algorithms may be developed to be predictive of the diffusion and corneal concentration mapping of specific formulations for variable thickness epithelium.

The present disclosure includes systems having controllers for providing various functionality to process information and determine results based on inputs. Generally, the controllers (such as the controller 120 described throughout the present disclosure) may be implemented as a combination of hardware and software elements. The hardware aspects may include combinations of operatively coupled hardware components including microprocessors, logical circuitry, communication/networking ports, digital filters, memory, or logical circuitry. The controller may be adapted to perform operations specified by a computer-executable code, which may be stored on a computer readable medium.

As described above, the controller 120 may be a programmable processing device, such as an external conventional computer or an on-board field programmable gate array (FPGA) or digital signal processor (DSP), that executes software, or stored instructions. In general, physical processors and/or machines employed by embodiments of the present disclosure for any processing or evaluation may include one or more networked or non-networked general purpose computer systems, microprocessors, field programmable gate arrays (FPGA's), digital signal processors (DSP's), micro-controllers, and the like, programmed according to the teachings of the exemplary embodiments of the present disclosure, as is appreciated by those skilled in the computer and software arts. The physical processors and/or machines may be externally networked with the image capture device(s) (e.g., the CCD detector 660, camera 760, or camera 860), or may be integrated to reside within the image capture device. Appropriate software can be readily prepared by programmers of ordinary skill based on the teachings of the exemplary embodiments, as is appreciated by those skilled in the software art. In addition, the devices and subsystems of the exemplary embodiments can be implemented by the preparation of application-specific integrated circuits or by interconnecting an appropriate network of conventional component circuits, as is appreciated by those skilled in the electrical art(s). Thus, the exemplary embodiments are not limited to any specific combination of hardware circuitry and/or software.

Stored on any one or on a combination of computer readable media, the exemplary embodiments of the present disclosure may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, and the like. Such computer readable media further can include the computer program product of an embodiment of the present disclosure for performing all or a portion (if processing is distributed) of the processing performed in implementations. Computer code devices of the exemplary embodiments of the present disclosure can include any suitable interpretable or executable code mechanism, including but not limited to scripts, interpretable programs, dynamic link libraries (DLLs), Java classes and applets, complete executable programs, and the like. Moreover, parts of the processing of the exemplary embodiments of the present disclosure can be distributed for better performance, reliability, cost, and the like.

Common forms of computer-readable media may include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read.

While the present disclosure has been described in connection with a number of exemplary embodiments, and implementations, the present disclosure is not so limited, but rather covers various modifications, and equivalent arrangements.

What is claimed is:

1. A system for generating cross-linking activity in an eye, the system comprising:
a light source for directing light to the eye to photoactivate a cross-linking agent applied to the eye; and
a feedback system configured to monitor a biomechanical strength of the eye in response to the photoactivation of the cross-linking agent, the feedback system including a perturbation system that applies a force to the eye and a characterization system that determines an effect of the force on the eye, the effect of the force providing an indicator of the biomechanical strength of the eye.

2. The system according to claim 1, further comprising a controller configured to:
analyze the indicator of the biomechanical strength of the eye from the feedback system;
determine, based on the indicator of the biomechanical strength of the eye, the photoactivation of the the cross-linking agent; and
direct the light to the eye, via the light source, to photoactivate the cross-linking agent according to a predetermined pattern.

3. The system according to claim 1, wherein the characterization system is configured to determine the effect of the force on the eye by measuring an amount of deformation caused by the force or a rate of recovery from the deformation.

4. The system according to claim 1, wherein the perturbation system applies intraocular pressure to the eye.

5. The system according to claim 1, wherein the perturbation system applies acoustic or ultrasound pressure waves to the eye.

6. The system according to claim 5, wherein the perturbation system applies shear supersonic ultrasound.

7. The system according to claim 1, wherein the perturbation system includes transducers configured for application to the eye.

8. The system according to claim 1, wherein the perturbation system includes a laser system.

9. The system according to claim 1, wherein the characterization system includes a phase shift interferometer.

10. The system according to claim 1, wherein the characterization system includes a corneal topography measurement system.

11. The system according to claim 1, wherein the characterization system includes a Scheimpflug system.

12. The system according to claim 1, wherein the characterization system includes an ocular coherence tomography system.

13. A method for generating cross-linking activity in an eye, the method comprising:
directing light to the eye, via a light source, to photoactivate a cross-linking agent applied to the eye; and
monitoring a biomechanical strength of the eye, via a feedback mechanism, in response to the photoactivation of the cross-linking agent, wherein monitoring the eye includes applying a force to the eye and determining an effect of the force on the eye, the effect of the force providing an indicator of the biomechanical strength of the eye.

14. The method according to claim 13, further comprising:
analyzing the indicator of the biomechanical strength of the eye from the feedback system;
determining, based on the indicator of the biomechanical strength of the eye, the photoactivation of the the cross-linking agent; and
direct additional light to the eye, via the light source, to photoactivate the cross-linking agent according to a predetermined pattern.

15. The method according to claim 13, wherein determining the effect of the force on the eye includes measuring an amount of deformation caused by the force or a rate of recovery from the deformation.

16. The method according to claim 13, wherein applying the force includes applying intraocular pressure to the eye.

17. The method according to claim 13, wherein applying the force includes applying acoustic or ultrasound pressure waves to the eye.

18. The method according to claim 17, wherein applying the force includes applying shear supersonic ultrasound.

19. The method according to claim 13, wherein applying the force includes activating transducers configured for application to the eye.

20. The method according to claim 13, wherein applying the force includes applying a laser to the eye.

21. The method according to claim 13, wherein determining the effect of the force on the eye includes determining the effect with a phase shift interferometer.

22. The method according to claim 13, wherein determining the effect of the force on the eye includes determining the effect with a corneal topography measurement system.

23. The method according to claim 13, wherein determining the effect of the force on the eye includes determining the effect with a Scheimpflug system.

24. The method according to claim 13, wherein determining the effect of the force on the eye includes determining the effect with an ocular coherence tomography system.
